(19) United States
(12) Patent Application Publication
(10) Pub. No.: US 2023/0218379 A1
(43) Pub. Date: Jul. 13, 2023

(54) An auxetic structure, a support structure, a method of preparing an auxetic structure, and use of a cellulosic material

(71) Applicant: AALTO-KORKEAKOULUSÄÄTIÖ SR, Aalto (FI)

(72) Inventors: Jani Kuula, Aalto (FI); Eija Raussi-Lehto, Aalto (FI); Rubina Ajdary, Aalto (FI); Orlando Rojas, Aalto (FI); Guillermo Reyes Torres, Aalto (FI)

(21) Appl. No.: 17/928,942

(22) PCT Filed: Mar. 19, 2021
(86) PCT No.: PCT/FI2021/050198

§ 371 Date: Dec. 1, 2022

(30) Foreign Application Priority Data
Jun. 1, 2020 (FI) 20207097

(51) Int. Cl. A61F 2/00; A61L 31/04; A61L 31/14; A61L 31/10

(52) U.S. Cl. CPC A61F 2/0045; A61L 31/042; A61L 31/146; A61L 31/10; A61F 2240/001; B33Y 80/00

(57) ABSTRACT

According to an example aspect of the present invention, there is provided an auxetic structure, comprising or consisting of cellulosic material, preferably comprising nanostructured or microstructured cellulose on at least one of its surfaces or consisting of nanostructured or microstructured cellulose and optionally other cellulosic material.

### FIELD

The present invention relates to support structures for mammalian tissues and organs, more particularly to for example pelvic organ support meshes and urethra support structures.

### BACKGROUND

Prolapse is associated with defective and failing pelvic floor tissues, which generates problems with pelvic organs (bladder, urethra, vagina, uterus, cervix, rectum, etc.). Typical negative effects include discomfort caused by weight, walking, sitting, urinating difficulty, pain, infections and odour. Because of the sensitive nature of prolapse, help is often sought after only at a stage when the symptoms are severe. According to studies, one in five women develops prolapse, with some estimates indicating that up to 40% of women are affected by this condition. Risk factors linked to such suffering from pelvic organ prolapse include childbirth, aging, obesity, constipation and heavy physical labour. Different types of pelvic organ prolapse (POP) exist. Prolapse is classified according to the affected organ and its descending part. Not all POP cases need an operation to fix the organs, but a large number of women go through a surgical POP operation each year. The lifetime risk for a female to undergo prolapse surgery is about 10% in Western Countries and more than 1 million surgical operations are performed annually because of POP condition.

Stress urinary incontinence (SUI) is a leakage of urine during moments of physical activity that increases abdominal pressure, such as coughing, sneezing, laughing, or exercise. SUI is the most common type of urinary incontinence in women. SUI can be treated surgically by placing a support mesh or sling.

Pelvic organ prolapse and stress urinary incontinence (SUI) can be treated operatively by a variety of methods. The procedures may be vaginal or abdominal. Many of the surgeries are carried out by using a supporting mesh as the main solution. The mesh is used to lift the loose tissue to its original position. POP surgery, as well as SUI surgery, is the only area of gynaecological surgery that is expected to grow significantly in the near future because of the aging population. The material used in known gynaecological meshes is synthetic and comprises either polypropylene, polyester or PTFE. However, U.S. Food and Drug Administration (FDA) reported in 2008 and 2011 about severe mesh-related complications. FDA stated that these problems with meshes are not rare, with 65% of patient considered in the groups being affected. As a result, the FDA banned the sales of several prolapse mesh products in 2016 and 2019. In the year 2016, FDA reclassified the meshes to the highest regulation class. The materials that are in current use are not biologically inert. For example, the synthetic polymers in use trigger a wide variety of adverse responses, including inflammation, fibrosis, calcification, thrombosis, or infection.

Surgical treatment of SUI has been described in Ulmsten U, Falconer C, Johnson P et al., A multicenter study of tension-free vaginal tape (TVT) for surgical treatment of stress urinary incontinence, Int Urogynecol J Pelvic Floor Dysfunct 1998; 9:210-3. An unmelted PP mesh tape is placed under the center of the urethra.

Ford A A, Rogerson L, Cody J D et al., Mid-urethral sling operations for stress urinary incontinence in women, Cochrane Database Syst Rev 2015; CD006375, describes retropubic (TVT) and transobturatorial (TOT, TVT-O) surgeries in the treatment of stress urinary incontinence. Mid-urethra slings (MUS) have almost completely displaced other surgeries previously used.

Cellulosic materials and fibres have been studied as alternatives to synthetic ones.

Cellulose is comprised of highly crystalline structure and does not dissolve in water and many organic solvents. However, a variety of solvents are already available to dissolve cellulose, including NaOH/water solutions in a limited range of low NaOH concentrations and at low temperatures. Aqueous NaOH solutions for dissolving cellulose are an attractive pathway for cellulose regeneration due to the low environmental impact, low cost, and ease of processability. Since the efforts in cellulose dissolution already in the 1930s, several additives and co-solvents have improved related alkali dissolutions properties. Davidson et al. (1937) reported cellulose dissolution in a mixture of ZnO/NaOH. In their work, they used NaOH concentrations of 2-4 mol/l with the addition of ZnO in the range of [0-0.178] ZnO/NaOH mass ratio, achieving a maximum solubility of oxidized cellulose of 8% w/w. The motivation behind cellulose dissolution has been to chemically modify the material in a homogeneous environment and to process the material to form new structures, such as filaments, films and even three-dimensional structures.

Various methods to regenerate cellulosic fibres or filaments are known.

Production of cellulose filaments typically involves coagulation of cellulose in a non-solvent (anti-solvent) coagulation bath. Previous to this step, a stable dope is prepared in the processing temperature to avoid gelation and thickening of the dope. Coagulation is a mass transport driven process, where the critical parameters for these alkali aqueous-based systems are mainly the dope concentration, dope rheology, and the coagulation bath conditions. Low concentrations and temperatures will hinder the coagulation process; contrarily, high temperatures and acid concentrations will produce soft filaments with high porosity. The mechanical performance of the filaments is optimized based not only on the heat and mass transfers involved in the coagulation processes but also on the gel hydromechanical properties, such as density and alignment. During the dope extrusion and the filament drawing, the shear forces create extensional forces that can effectively orient the structure. Thus, conforming well-aligned filaments has the potential to enhance the strength and toughness. A well-balanced dope should not be too soft because the fibrils alignment would be lost, neither too rigid to facilitate the extrusion and drawing steps.

Various mesh structures have been studied in recent years.

Katrina M. Knight et al., Preventing Mesh Pore Collapse by Designing Mesh Pores With Auxetic Geometries: A Comprehensive Evaluation Via Computational Modeling, Journal of Biomechanical Engineering May 2018, Vol. 140, 051005-1, discloses synthetic meshes with a macrostructure that results in auxetic behavior.

Fang-Fang Ai, Meng Mao, Ye Zhang, Jia Kang, Lan Zhu, Experimental study of a new original mesh developed for pelvic floor reconstructive surgery, Int. Urogynecol. J. (2020) 31:79-89, describes bacterial cellulose meshes for pelvic floor reconstruction.

The present invention aims at overcoming at least part of the disadvantages in the known solutions.

### SUMMARY OF THE INVENTION

The invention is defined by the features of the independent claims. Some specific embodiments are defined in the dependent claims.

According to a first aspect of the present invention, there is provided an auxetic structure comprising or consisting of cellulosic material.

Various embodiments of the first aspect may comprise one or more features from the following bulleted list:
The cellulosic material comprises nanostructured or microstructured cellulose on at least one of its surfaces, or consists of nanostructured or microstructured cellulose and optionally other cellulosic material.
The auxetic structure comprises a mesh-like overall structure.
The auxetic structure comprises a knitted or woven auxetic structure.
The auxetic structure comprises a moulded auxetic structure having openings extending through the structure.
The auxetic structure comprises a guiding mesh or guiding filaments made of a thermoplastic material and at least partially, preferably entirely coated with microstructured or nanostructured cellulose.
The cellulosic material comprises or consists of bacterial nanocellulose.
The thickness of the coating is at least 10 nm, such as in the range 20 to 500 nm, for example in the range 50 to 150 nm.
The auxetic structure comprises cellulosic material, such as cellulose nanofibrils or cellulose filaments, obtained by a spinning process.
The auxetic structure comprises filaments or a mesh obtained from a cellulosic material by a printing process, such as 3D printing or direct ink writing, preferably from a gel or a suspension comprising cellulosic material.
The structure has a negative Poisson's ratio and consequently exhibits lateral expansion when subjected to tension.
The structure comprises nanocellulose, such as wood-based nanocellulose or bacterial nanocellulose, and polyolefin.
The structure consists of nanocellulose.
The nanocellulose comprises or consists of cellulose produced by bacteria, which is preferably in the form of nanofibrils.
The nanocellulose comprises or consists of wood-based cellulose which has been disintegrated to nanostructured cellulose.
The structure comprises openings that are configured to expand under load.
The auxetic structure has a Young's Modulus of at least 700 MPa in wet condition.
The auxetic structure has tensile strength of at least 1.5 MPa in wet condition.
The auxetic structure is for use in the treatment of pelvic organ prolapse, urinary incontinence, breast reconstruction, hernias, or fecal incontinence.

According to a second aspect of the present invention, there is provided a support structure, comprising the auxetic structure according to the first aspect, wherein said support structure is configured to support a tissue or an organ of a mammal, such as a human or a dog.

Various embodiments of the second aspect may comprise one or more features from the following bulleted list:
The support structure is a pelvic organ prolapse mesh, a urinary incontinence sling or tape for a man or a woman, a breast reconstruction support structure, a hernia mesh or a fecal incontinence support structure.
The support structure is a support mesh, a support sling or a support tape.

According to a third aspect of the present invention, there is provided a method of preparing an auxetic structure, comprising: forming an auxetic structure comprising or consisting of a cellulosic material, or providing an auxetic guiding structure, and applying or growing a cellulosic material on at least a part of a surface of the guiding structure.

Various embodiments of the third aspect may comprise one or more features from the following bulleted list:
The cellulosic material comprises or consists of nanostructured and/or microstructured cellulose.
The auxetic guiding structure is prepared in a form of a guiding mesh by knitting from filaments or by printing.
Said filaments comprise or consist of hydrophobic filaments, such as synthetic hydrophobic filaments, for example filaments made of a thermoplastic material, such as nylon, polypropylene, polyethylene, polyurethane, polycaprolactone, polyethylene terephthalate or a combination thereof.
Said filaments comprise of consist of polypropylene filaments.
Said guiding mesh is prepared by means of a 3D printing method from a biodegradable thermoplastic material, such as PLA.
Before said applying or growing step, said guiding structure is treated by a priming treatment, such as a hydrophilization treatment, an oxidizing treatment, a plasma treatment, or a protein adsorption treatment, and as a result of said priming treatment, the bonding between the cellulosic material and a surface of the guiding structure is improved.
Said auxetic guiding structure comprises hydrophilic filaments, such as filaments made of cellulose nanofibrils by a spinning process.
Said applying or growing step comprises introducing a bacterial nanocellulose culture medium on a surface of said auxetic guiding structure, and allowing said culture medium to grow on said auxetic guiding structure.
Said forming comprises: dissolving a cellulosic material; regenerating the dissolved cellulosic material to obtain a cellulosic gel or cellulosic fibres; preparing the auxetic structure from said gel or fibres, preferably by knitting or printing.
Said forming comprises moulding an auxetic structure of a cellulosic material comprising or consisting of nanostructured and/or microstructured cellulose.
The method comprises providing a positive master mould exhibiting an auxetic shape, preparing a negative mould from a thermoplastic polymeric material by using the positive master mould, filling the negative mould with said cellulosic material comprising or consisting of nanostructured and/or microstructured cellulose, to prepare a corresponding positive structure, which is the auxetic structure.
Said filling comprises applying a bacterial nanocellulose culture medium into said negative mould and allowing said culture medium to grow and fill the mould.

According to a fourth aspect of the present invention, there is provided use of a cellulosic material, such as nanostructured or microstructured cellulose, preferably bacterial nanocellulose, in a coating of or as a material of an auxetic structure configured for use as a support structure for a tissue or an organ of a mammal, such as a human.

According to a fifth aspect of the present invention, there is provided a method of treating a mammal, comprising implanting a support structure according to the second aspect into a body of the mammal to support a tissue or an organ of the mammal.

### Advantages of the Invention

Some embodiments of the present invention increase biocompatibility of tissue support structures intended for use inside a human body.

Some embodiments of the present invention may reduce infection risks involved in the use of pelvic organ support meshes and like.

Some embodiments of the present invention provide support structures that have improved mechanical properties under load.

Some embodiments of the present invention provide customizable support structures obtainable for example by 3D printing and bioprinting methods.

### BRIEF DESCRIPTION OF THE DRAWINGS

FIG. 1 illustrates preparation of an auxetic BNC mesh by mold guiding approach in accordance with at least some embodiments of the present invention;

FIG. 2 shows a 3D printed hydrophobic polymer in the form of an auxetic mesh in accordance with at least some embodiments of the present invention;

FIG. 3 shows scanning electron micrographs of a BNC coating on the surface of a hydrophobic mesh pre-treated by a surface interaction enhancing method in accordance with at least some embodiments of the present invention;

FIG. 4 displays development of an auxetic CNF 3D printed mesh in accordance with at least some embodiments of the present invention; and

FIG. 5 illustrates preparation of a cellulosic dope and regeneration of cellulosic filaments in accordance with at least some embodiments of the present invention.

### EMBODIMENTS

### Definitions

In the present context, the term “bacterial cellulose” comprises cellulose produced by bacteria.

In the present context, the term “bacterial nanocellulose” comprises nanostructured cellulose produced by bacteria.

In the present context, the term “nanocellulose” comprises nanostructured cellulose.

In the present context, the term “microcellulose” comprises microstructured cellulose.

In the present context, the term “mesh” comprises a two-dimensional or three-dimensional porous structure that comprises a web-like pattern or construction.

In the present context, the term “auxetic structure or material” refers to a structure or material that has a negative Poisson's ratio.

In the present context, the term “single-filament structure” refers to a structure obtained by for example knitting with a single strand of yarn or filament and that typically shows uniformly shaped openings. The term “double-filament structure” refers to a structure obtained by knitting with two strands of yarn or filament.

We have surprisingly observed that many advantages may be achieved by use of auxetic organ support structures that comprise a cellulosic material.

The present invention provides an auxetic structure comprising cellulosic material, such as nanocellulose and/or microcellulose.

In preferred embodiments, the auxetic structure is part of a support structure for mammalian tissue, such as human tissue.

For example, the support structure may be any of the following: a pelvic organ prolapse mesh (transvaginal and sacrocolpopexy approach), urinary incontinence sling or tape for men or women, a breast reconstruction support structure, a support structure for treating hernias, or a fecal incontinence support structure.

Some embodiments of the present invention provide a nanocellulose net or mesh with or without nylon or polyolefin backbone, with mono or multifilament auxetic network pattern, optionally with tissue growth enhancing substance release to treat pelvic organ prolapse.

In some embodiments, the auxetic structure comprises a cellulosic material and a thermoplastic polymeric material in combination.

In a preferred embodiment, the cellulosic material comprises or consists of nanocellulose, such as bacterial nanocellulose, plant-based nanocellulose, such as wood-based nanocellulose, or algae-based nanocellulose or a combination thereof. Preferably the cellulosic material is a biocompatible material comprising or consisting essentially of cellulose.

In some embodiments, the thermoplastic polymeric material comprises or consists of a polyolefin, such as nylon, polypropylene (PP) and/or polyethylene (PE), polyurethane (PU), polycaprolactone (PCL) or polyethylene terephthalate (PET), or a combination thereof.

In some embodiments, the support structure is in the form of a mesh or a tape or a sling. The support structure may be formed by a bottom-up or top-down manufacturing method.

In some embodiments, the auxetic structure may be entirely formed of a cellulosic material comprising or consisting of nanostructured and/or microstructured cellulose. The structure may be prepared for example by moulding or by printing from a cellulosic material.

In one embodiment, the auxetic structure may comprise a core structure or a guiding structure which has been at least partly coated by a surface layer. In one embodiment, the composition of the core structure is different from the composition of the surface layer. In this alternative, the surface layer preferably comprises or consists of nanostructured and/or microstructured cellulose.

In one embodiment, the core structure is made of at least one thermoplastic polymeric material.

In one embodiment, the surface layer is made of a cellulosic material, such as nanocellulose, microcellulose or a combination thereof.

Advantageously, at least 50%, such as at least 90%, of all surfaces of the core structure are coated by a cellulosic material in order to provide biocompatibility. In this way the biocompatibility of the support structure as a whole may be increased in comparison to support structures that do not contain such a coating. This effect may be observed particularly in the case of using nanocellulosic materials as the cellulosic material of the coating.

By the term “biocompatibility” it is referred to the capability of an article or a material implanted in the body to exist in harmony with tissue without causing deleterious changes.

In one embodiment, the thermoplastic polymeric material forms a core structure on which the cellulosic material is applied or grown. For example, the thermoplastic polymeric material may first be printed to provide an auxetic guiding structure, such as a mesh, on which cellulosic material is provided, for example by growing or depositing.

In an advantageous embodiment, at least a part of the auxetic structure, for example a guiding structure of the auxetic structure, is formed by a printing method, such as a 3D printing method. The advantage of using a 3D printing method is the ease of developing customizable structures, which might be challenging to make with other processing techniques. 3D printing method is also considered as a fast and cheap method with minimum waste in use of raw material.

In the following we explain in more detail embodiments in which various cellulosic materials are used as materials of the auxetic structure.

Cellulose and bacterial nanocellulose (BNC)

Cellulose is the most abundant natural polymer on the earth and comprises the basic structural matrix of the cell wall in almost all plants. In addition to plants and trees, cellulose is also produced by few other resources including algae (Valonia) and some bacteria groups such as Acetobacter, Sarina, and Agrobacterium. Celluloses produced by different resources all share similar molecular formula of (C₆H₁₀O₅)ₙ, however, they are used for different purposes considering their compositional and morphological differences. Cellulose from wood and plants are accompanied by lignin, hemicelluloses, and pectin. However, cellulose produced by bacteria is highly pure, has a higher cellulose production rate and lower energy processing compared to plants. In addition to purity, nanocellulose from bacteria (BNC) has remarkably higher tensile properties due to the nanofibrils entanglement as a web-like network structure. When compared with plant-based cellulose, BNC has higher degree of polymerization, higher specific area, high water holding capacity (as high as hundreds of times its dry weight in water), and ease of production. High porosity combined with high surface area makes BNC an ideal material for interacting with active compounds and medicine. BNC is also highly biocompatible. BNC is currently produced by three methods: static, shaking, and bioreactor methods.

In the static method, that is also used in some embodiments of the present invention, a gelatinous membrane of cellulose grows on the surface of static culture medium, where it receives oxygen from the top and nutrition from the culture medium. The condition of culture medium has a determining impact on the properties of BNC (pH, nutrition, oxygen delivery, temperature, and bacterial strain). Acetobacter xylinum, also known as G. xylinum, has highest BNC production rate compared to other bacteria types. Such non-photosynthetic, aerobic bacterial strain converts glucose and other organic compounds to cellulose within a few days. According to previous studies, 108 glucose molecules are converted to cellulose by one bacterium.

Auxetic Structures

Poisson's ratio defines the relative change in natural dimension of an object under directional load according to the following equation:

Poisson's ratio=ε_{y}/εₓ,

wherein ε_{y} is the transverse strain and εₓ is the axial strain.

Most known materials have a positive Poisson's ratio (conventional materials). Structure of conventional materials shrinks when stretched and expands when compressed. On the other hand, Negative Poisson's ratio (NPR) structures, also known as auxetic structures, exhibit lateral expansion and contraction when stretched and compressed respectively. NPRs occur rarely in nature (α-cristobalite crystalline silicate, zeolites, and some soft tissues), however, these structures have been developed synthetically with wide range of materials and structures.

The structural changes under load are essential for biomedical meshes. The void space in mesh structures are often designed and reported under no-load state. However, the void geometry highly alters according to the applied load. The void openings are often fully collapsed while bearing the load of pelvic floor. Shrinkage of the void openings to below 1 mm challenges the tissue ingrowth, and possibly leads to inflammation, pain, and an increased risk of bridging fibrosis. It is important to maintain the void openings to better integrate the mesh with host tissue and minimize the challenges caused by geometrical changes in the mesh.

As an alternative to a pure BNC mesh, BNC can also be applied as a coating around a guiding mesh, which is typically comprised of hydrophobic or hydrophilic filaments. Such a guiding mesh ensures an adjustable mechanical strength as well as provides a biocompatible mesh surface. Such biocompatibility is highly advantageous, as upon use, the mesh surface will be in contact with a tissue.

Monofilament or Yarn Structures

In some embodiments, the mesh has a monofilament or a single-filament structure. Single filament meshes are advantageous in the sense that they may decrease the risk of infection when compared to the known multifilament PP mesh structures in which interstices with size less than 10 microns may host bacteria and immune cells. Some embodiments of the present invention provide monofilament structures or yarn structures covered with BNC to reduce bacterial or other source of infection.

Tissue Growth Enhancing Substance Release

Biocompatibility, biodegradability, being non-toxic, and high porosity are important characteristics of BNC as a potential carrier in biomedical applications. In some embodiments of the present invention, these properties are utilized, and a drug or active substance is loaded into the mesh structure, to be later released into human body.

While the use of BNC is advantageous, various other cellulosic materials may alternatively or in addition be used either in the form of self-standing auxetic structures or as coatings on auxetic guiding or core structures.

Next, exemplary approaches are described:

Approach 1: Mould guiding towards neat bacterial nanocellulose meshes grown in negative auxetic moulds
Approach 2: Filament guiding using hydrophobic and hydrophilic core filaments, to provide 3D printed or knitted auxetic meshes with bacterial nanocellulose coating
Approach 3: 3D printed hydrophilic mesh with auxetic design
Approach 4: Use of regenerated cellulosic filaments and gels

Approach 1: Mould Guiding Towards Neat Bacterial Nanocellulose Meshes Grown in Auxetic Negative Moulds.

Preparation of Bacterial Nanocellulose (BNC)

The nutrition for the culture medium includes glucose, yeast extract, peptone and disodium phosphate (Na₂HPO₄) in water. To prepare the culture medium the recipe below was followed. First, powders of glucose, yeast extract, peptone, and Na₂HPO₄ were mixed with dry mass ratio of (8:2:2:1). Then MilliQ-water was added to the bottle (For example: enough water to reach 1 liter volume to 20 g glucose, 5 g yeast extract, 5 g of peptone, 2.5 g of Na₂HPO₄). The pH of the medium was adjusted to pH 4.5 with citric acid. Then, the bottle was sterilized in an autoclave for 15 min at a temperature of 120° C. followed by a cooling down step to room temperature. Later, the strain (bacteria in skim milk) was added to the bottle and shaken to distribute properly. The medium was left static for the bacterial cellulose to grow up to 10 to 14 days at a temperature of 28° C. in the incubator. After two weeks, the fermented bacterial cellulose pellicle was washed by deionized water several times and the pellicle was left in deionized water for one day. The water was changed several times within the day for removing the chemicals of the growing medium. The bacterial cellulose pellicle was purified by boiling it in 0.1 M NaOH, 60° C., for 4 h and thereafter repeated boiling in deionized water.

Formation of a pure BNC mesh with an auxetic structure is discussed in the next section.

Auxetic Structures and Auxetic Negative Moulds

To address the challenges with the mechanical properties, in this exemplary approach we use a negative auxetic mould. The choice of auxetic pattern is according to the mechanical and structural points of view, enlarging the void space under load, and ease of 3D printability by Fused deposition moulding (FDM).

The desired auxetic structure was first designed by Solid Work and then 3D printed by an Ultimaker 2 FDM 3D printer in 10 cm to 10 cm to 4 cm (height) using polylactic acid (PLA) filaments (positive moulds). Mold Star TM 30 silicone rubber (Part A and B) was used to form the negative mould. Equal mass ratio of part A and part B were added together and mixed properly and fully filled the positive moulds. The full curing time in the silicone rubber is about 6 hours, however, partial hardening initiates in about 30 minutes after mixing parts A and B.

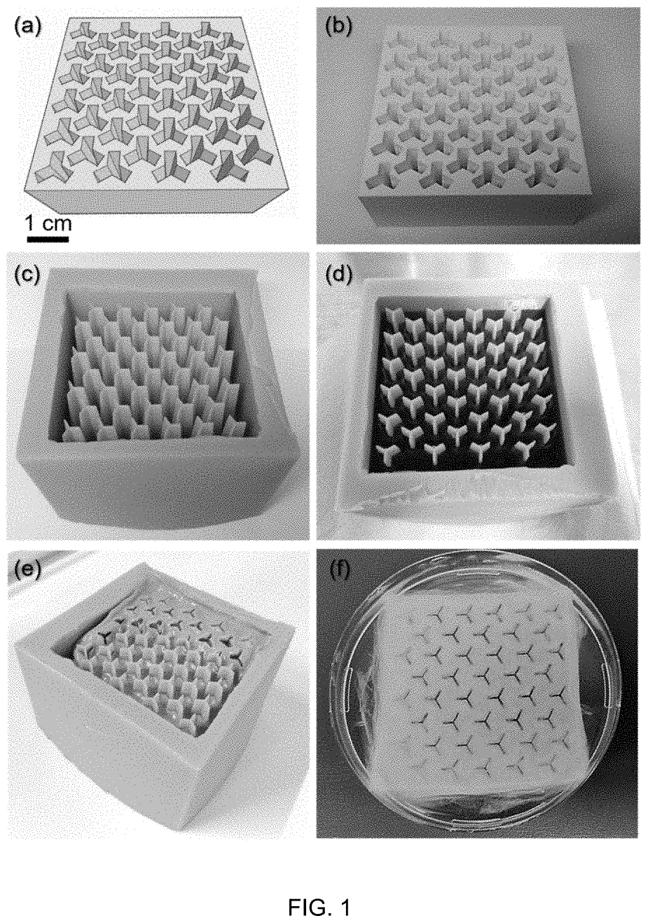

The steps of preparing the silicone moulds from 3D models to negative moulds are displayed in FIG. 1. The negative moulds were filled with the BNC culture medium and sealed with paraffin for 10 days for BNC to grow on the surface in the form of an auxetic mesh. The auxetic BNC mesh had the open void percentage of 30-70% depending on the tension applied to the mesh and the auxetic design. The mechanical properties of the never-dried BNC mesh improved by increasing the growth time from 7 to 14 days as indicated in Table 1.

TABLE 1
Mechanical properties of never-dried BNC mesh. The values in
parentheses are standard deviations of the respective values.

| | Modulus | | |
|---|---|---|---|
| Sample | [GPa] | Tensile Strength [MPa] | Strain [%] |
| BNC 7 days | 1.7 (0.3) | 179 (24) | 2 (0.17) |
| BNC 10 days | 5.3 (0.8) | 518 (34) | 2.9 (0.24) |
| BNC 14 days | 8.6 (0.7) | 787 (71) | 6.2 (0.29) |

FIG. 1 shows the mesh development by the mould guiding approach. FIG. 1(a) illustrates a 3D model of a negative auxetic mold. FIG. 1(b) illustrates a 3D printed negative auxetic mould. FIG. 1(c) illustrates a casted positive silicon mould. FIG. 1(d) illustrates a bacteria culture medium in silicon mould (day 0). FIG. 1(e) illustrates a BNC mesh on the surface of the culture medium (day 10). FIG. 1(f) illustrates a purified auxetic BNC mesh in size of 10 cm×10 cm.

Approach 2: Filament Guiding Using Hydrophobic and Hydrophilic Core Filaments with BNC Coating

In this exemplary approach, either hydrophobic or hydrophilic filaments were used as guiding structures when growing bacterial nanocellulose onto their surface. Such guiding structures function as core structures on which the bacterial nanocellulose is grown with adjustable thickness by controlling the bacteria cellulose growth time.

Hydrophobic filaments (PP, PE, PU, PCL and nylon) may be used as core filaments or as core meshes.

For this purpose, the core polymer should fulfil certain mechanical properties to perform under load to lift the prolapsed organ. We have observed that according to the nature of core polymer (PP, PE, PU, PCL and/or nylon, for example nylon, PP or PE) enhancement of the surface energy and hydrophilization of the surface improves the interaction of the filament with BNC coating. This can be achieved by priming the hydrophobic mesh with plasma treatment/ozone or with coupling biomolecules such as soy proteins.

The hydrophobic polymers are integrated or shaped into a mesh form.

In one embodiment a 3D printed hydrophobic mesh with auxetic design is produced.

An auxetic 3D mesh model was first imported to the 3D printer, and the parameters were adjusted according to the polyolefin or polymer input, including printing speed, extrusion pressure and temperature, and structure infill. The mesh structure is formed by extruding the molten polymer with an Ultimaker 2+3D printer equipped with a thermoplastic toolhead. A priming surface treatment is performed on the ready-made 3D printed mesh.

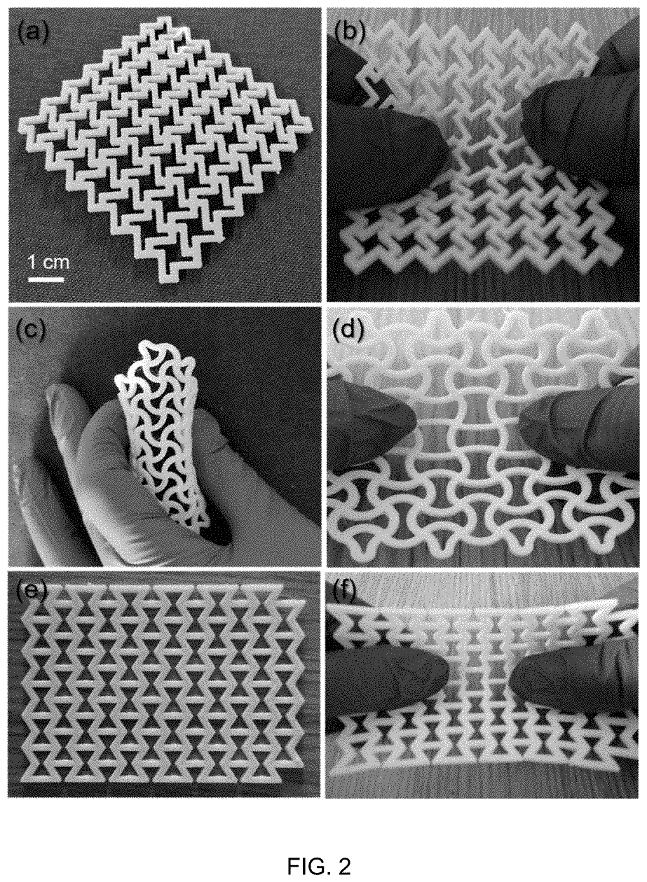

FIG. 2 illustrates (on the left side) 3D printed hydrophobic auxetic meshes in various geometries: (a) square, (c) round, and (e) re-entrant hexagon. The expansion of the mesh and the structure under tension is shown (on the right side) respectively: (b) square, (d) round, and (f) re-entrant hexagon.

Knitting of Filaments to Provide Auxetic Effect

The filaments may be knitted following a geometrical auxetic model to benefit from the expansion of openings under load of a prolapsed organ.

Various surface treatments may be applied to enhance the surface interaction of a hydrophobic mesh with a BNC coating.

One embodiment involves priming the hydrophobic mesh with plasma treatment/ozone.

The low surface energy in hydrophobic polymers such as PP and PET cause challenges with proper adhesion of additional molecules. Gas-phase surface modifications, such as plasma treatment and exposure to ozone-generating UV light, oxidize the surface of the polymer and make it more hydrophilic/wettable. Combination of UV/air and ozone treatment for 10 minutes decreases the advancing and receding contact angle of PP by about 30 to 40°. The decrease in the contact angle value reaches a plateau after 60 min exposure to the treatment. Similar treatment on PET decreased the advancing contact angle to 50° after 10 minutes. PP and PET require almost similar exposure time to reach significant surface oxidation. However, PP modification is mainly governed by oxygen atoms formed during exposure of ozone to UV light. While, UV light directly affects PET and causes surface modification by chain scission.

Another embodiment involves priming the hydrophobic mesh by coupling of biomolecules such as soy protein.

Protein adsorption is another method to address the low surface energy and wettability of hydrophobic filaments (such as PP and PET filaments). This technique activates the material surface with hydrophilic groups and enhances the interaction with water. Protein adsorption of the surface is affected by the solid substrate, protein structure, physical-chemical bonding variables (hydrogen bonding, van der Waals, as well as electrostatic forces). Proteins from soybean may be attached on the surface of PP films following a simple immersion technique. The protein affinity to PP surface may be considerably enhanced by a pre-treatment step that includes immersion of PP films in 2-propanol (20 minutes) followed by dipping in a solution of a cationic surfactant (1 mg/mL DODA) for 30 minutes. Afterwards, the mesh was rinsed with a phosphate buffer solution at pH 7.4 and immersed in a protein solution for a duration of minimum 1 hour. The physical adsorption of protein to PP hydrophobic surface remarkably changes the wettability of the films and increases the surface energy for further treatments.

Finally, a BNC coating may be applied on the mesh.

As the last step, the mesh comprised of treated or primed hydrophobic mesh is dipped in bacterial cellulose culture medium (a similar culture medium as in Approach 1), lifted and left in high relative humidity at 28° C. in incubator for BNC to grow and cover the mesh. The plasma/ozone or the protein adsorption enhance the filaments wettability as well as the interaction with bacteria containing culture medium. The thickness of BNC is directly controlled by the growth time and feeding the existing bacteria on the surface of the mesh by adding autoclaved 20 wt-% glucose solution.

After attaining the full coverage of filaments in the mesh by BNC in desired thickness, the BNC-coated mesh is purified by boiling in water and 0.1 M NaOH at 60° C., for 4 h followed by several rounds of subsequent boiling in deionized water.

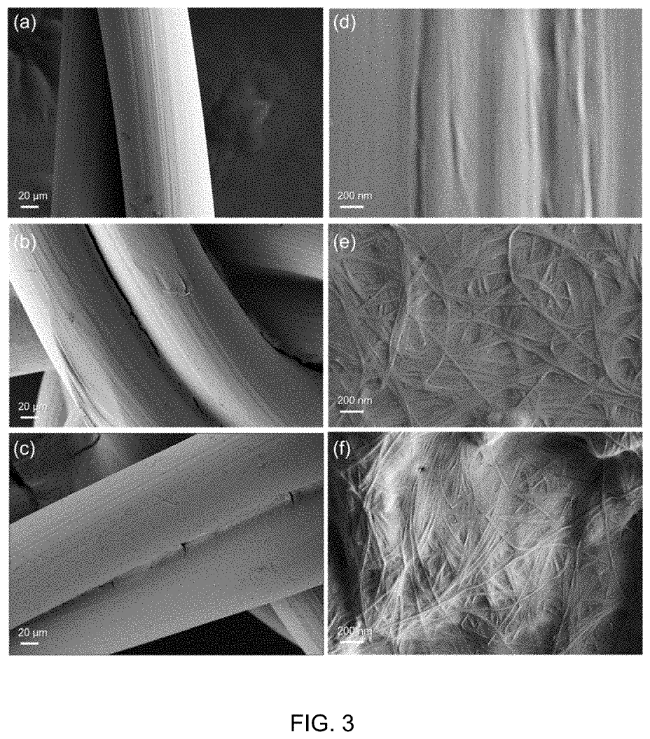

FIG. 3 shows scanning electron microscope images of BNC coating on hydrophobic treated and non-treated substrates. FIG. 3(a) shows a non-treated filament. FIG. 3(b) shows a filament treated with UV light. FIG. 3(c) shows a filament treated by coupling biomolecules from soy protein. FIGS. 3(a) to 3(c) are in 200× magnifications and FIGS. 3(d) to 3(f) in 25000× magnifications.

Hydrophilic Filaments

An aqueous suspension of cellulose nanofibrils (CNF) may be assembled into one-dimensional filaments by a wet spinning process. In this method the CNFs are extruded through a spinneret into a coagulation bath including an antisolvent. The formulation of the spinning dope, shear rate, and drawing ratio are essential factors in both formation and properties of filaments. Wet spun filaments typically comprise at least partially aligned nanofibrils with traces of orientation caused by extensional flow.

Addition of nanochitin and/or chitosan, which are biopolymers obtained from marine biomass, to the CNF suspension before extrusion may provide the filaments with antimicrobial properties. Nanocellulose and chitosan filaments were developed through wet spinning of core and shell structures in HCl 0.01 M (pH 2). Negatively charged nanocellulose (−47 mV) was placed in the shell, providing mechanical strength and support for the positively charged chitosan (+42.8 mV) in the core. The mechanical properties of the filaments are shown in Table 2.

TABLE 2
Mechanical properties of wet spun nanocellulose and nanocellulose-
chitosan filaments in dry conditions. The values in parentheses
are standard deviations of the respective values.

| | Modulus | Tensile Strength | Tensile Strain |
|---|---|---|---|
| Sample | [GPa] | [MPa] | [%] |
| Nanocellulose (TOCNF) | 18 (3) | 135 (15) | 2.4 (0.9) |
| TOCNF-chitosan | 22 (6) | 163 (21) | 2.2 (0.8) |

The obtained CNF filaments are by nature hydrophilic and have a high affinity to water or water-based mediums. Bacterial nanocellulose may be grown on the surface of hydrophilic CNF filaments.

Knitting of Filaments to Provide Auxetic Effect.

Also in the case of hydrophilic filaments, the filaments may be knitted following a geometrical auxetic model to benefit from the expansion of openings under load of a prolapsed organ.

One method is to make a wrap knitted mesh that includes pillar stiches and inlay yarns. This knitting method is comprised of thin and ticker filaments of the same or a different material. As the mesh stretches, the openings formed by the thinner filaments wrap around the thicker ones and straightens it to create open voids in the overall structure.

Finally, a BNC coating may be applied on the mesh.

As the last step, the mesh comprised of hydrophilic filaments may be dipped in bacterial cellulose culture medium (a similar culture medium as in Approach 1), lifted and left in high relative humidity at 28° C. in incubator for BNC to grow and cover the mesh. The thickness of BNC may be controlled by the growth time.

After attaining the full coverage of filaments in the mesh by BNC in desired thickness, the BNC-coated mesh may be purified, for example by boiling in 0.1 M NaOH water solution at 60° for 4 hours followed by several rounds of subsequent boiling in deionized water.

Approach 3: 3D Printed Hydrophilic Mesh with Auxetic Design

In one embodiment, a 3D printed hydrophilic mesh with auxetic design is produced from nanocellulose.

An auxetic 3D mesh model was first imported to the 3D printer, and the parameters were adjusted according to the nanocellulose hydrogel input, including printing speed, extrusion pressure and nozzle diameter, and structure infill. The mesh structure is formed by direct ink writing of nanocellulose with a BIOX bioprinter equipped with a pneumatic printhead. A plastic petri dish was used as a substrate for the printed mesh.

An exemplary process is described next.

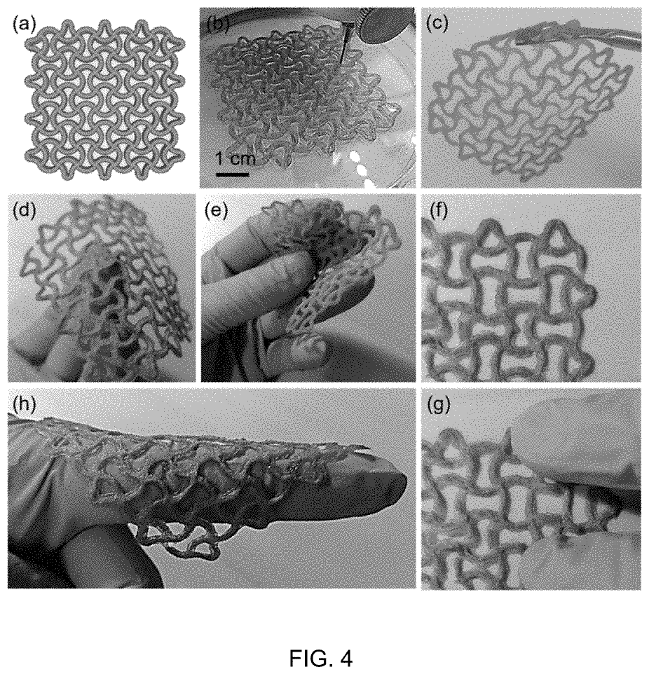

TEMPO-oxidized nanocellulose (TOCNF), produced through disintegration of never-dried, fully bleached wood fibers was used in 1.5 wt-% solid content to print auxetic mesh structures. A Cellink Bioprinter equipped with pneumatic print head was used to extrude CNF and deposit the layers to form a mesh. The system utilized a clear pneumatic 3 mL syringe and sterile blunt needle 18 G (needle size 0.84 mm). The needle size may be in the range 22 G (0.41 mm) to 15 G (1.3 mm). Printing parameters including the nozzle size, print head speed, and extrusion pressure were adjusted to achieve suitable conditions for 3D printing of CNF, according to the quality and fidelity of the 3D-printed structure. After 3D printing the mesh was frozen at −18° C. and then dried under vacuum for 48 h at −49° C. Then the mesh was crosslinked to enhance the mechanical strength and flexibility of the structure. The crosslinking was performed by immersing the mesh in 100 ml of glutaraldehyde 25 wt-% solution containing 100 μl of hydrochloric acid in room temperature as an acid catalysis. After one hour of immersion, the mesh was rinsed with DI water several times. The nanocellulose auxetic mesh is displayed in FIG. 4. The nanocellulose mesh was 94% porous structure and the Young's modulus and tensile strength of nanocellulose mesh was 0.7 GPa and 1.5 MPa respectively.

FIG. 4 illustrates the nanocellulose auxetic mesh. FIG. 4(a) shows the 3D model. FIG. 4(b) shows the 3D printed nanocellulose auxetic mesh in wet condition. FIG. 4(c) shows the nanocellulose auxetic mesh after sterilization with ethanol. FIGS. 4(d) and 4(e) illustrate the flexibility of the mesh after cross-linking with glutaraldehyde. FIGS. 4(f) and 4(g) illustrate the auxetic behaviour of the mesh and expansion of the structure under tension. FIG. 4(h) shows the mesh in wet condition. The size of the 3D printed mesh was 6 cm×6 cm.

Approach 4: Regenerated Cellulosic Filaments and Gels

In this embodiment, one-dimensional (1D) cellulose filaments are provided by a regeneration pathway with controlled rheology, cross-linking process, and high mechanical strength. The produced continuous filaments may be knitted in the form of a mesh to be used for POP, SUI, and hernia tissue support. In addition to a wet spinning process, the developed gel may be processed through a direct ink writing method to produce a 3D structure.

In the following we describe the preparation process of the filaments.

The present work is focused on dissolution in a NaOH 2.3M system with ZnO addition, maintaining a ZnO/NaOH mass ratio of 0.167. The cellulose (Avicel PH-101 microcrystalline cellulose, 50 μm particle size, DP 400, purchased from Sigma-Aldrich) (7% w/w) previously dried under vacuum (200 mbar, 60° C., 12 h) was added to a pre-cooled NaOH/ZnO aqueous solution at −5° C. This system was stirred (300 to 700 rpm) in a vessel with a cooling jacket (using water/propylene glycol 1:1), controlling the temperature at −5° C. for 24 h. After the stirring procedure, an opaque and viscous solution was obtained; this solution was collected and frozen at −17° C. for 12 h. The freezing step improves the NaOH hydrated shells' contact with the reactive hydroxyl groups of cellulose, improving the dissolution. As the final step, the solid dope was thawed using an innovative process for homogenizing and deaeration of the dope: The process was performed in a planetary centrifuge (THINKY AR-250 mixer, JAPAN), where the solid dope was thawed simultaneously under centrifugal forces (2000 rpm, 20 min) allowing to obtain a transparent and completely dissolved dope (as observed by optical microscopy) which had low viscosity of 465 mPa·s that is suitable for a spinning process.

The shear rheology of the dissolved cellulose was monitored. First, the linear viscoelastic region (LVR) of the cellulose dope is determined by an oscillation test where an amplitude sweep at constant frequency allows to determine the range where the elastic structure of the sample is not destroyed. This test, also called the fracture test, is necessary to establish the amplitude range where the complex modulus is constant; therefore the rheological properties of the dope can be measured. The dope exhibited stability at 10 rad/sec in a wide range of amplitudes. Therefore, 1% strain was selected to measure the rheological properties. The gelation kinetics was determined at 10 rad/sec and 1% strain measuring the gelation time, the time where the storage modulus (G′) became more extensive than the loss modulus (G″) or, in other words, the phase angle became smaller (tan δ<1). The master's plot was constructed by measuring eight different temperature points in the range of 15° C. to 40° C., and its kinetics were determined to follow Equation (1).

$$t = 1053 {e}^{\frac{- T}{8}} \qquad ( 1 )$$

In Equation (1), t is time in minutes for the gelation to occur, and Tis the dope temperature in Celsius degrees. This kinetic equation allows to predict the gelation time under the conditions explored. For instance, gelation at 10 rad/sec, 1% strain, and 25° C. occurs in about 1 hour.

Additionally, the frequency sweep test at room temperature (25° C.) showed that the dope initially exhibited a gel-like behaviour G′>G″ for frequencies in the range 0.1 to 50 rad/s and then displayed a liquid-like behaviour G′<G″ for frequencies above 60 rad/sec. This could be interpreted as in the initial pre-gelation stage where the interfibrillar interactions caused the shear stress at high frequencies and allowed to recover a liquid-like behaviour destroying metastable interactions. It is essential to point out that this only occurs before complete gelation, and this gives a hint for using the planetary centrifugal equipment during thawing. This result is also aligned with the viscosity test, where the dope exhibited a thinning behaviour exponentially falling from a zero shear rate viscosity of 1500 mPa·s until 465 mPa·s for shear rates approaching 100 s⁻¹.

The second frequency sweep test was carried out at 0.01% strain where the dope that exhibited a liquid-like behaviour (G′<G″) in the full range of angular frequencies from 0.1 to 100 rad/s displayed a solid-like behaviour. This occurred by adding 25% v/v of a solution containing 1% w/w of a Lewis acid such as ZnCl₂ or FeCl₃. Dopes that initially possessed a storage modulus around 1 Pa shifted to a modulus around 300 Pa, with G′>>G″ for all frequencies. These last results suggested that this type of acids could be used during or after the coagulation process to improve the interfibrillar connectivity.

The previously prepared cellulose dope is collected in a 50 ml Luer lock syringe to be extruded using a dispensing needle (Ramé-Hart Instrument CO, gauge 21 inner diameter Φₒ=508 μm). The wet spinning system used a pump (CHEMYX, model NEXUS 6000, and CHEMYX, model FUSION 6000, USA) to extrude the dope into an acid bath at a volumetric speed of Q=0.1 ml/min. As an initial reference assuming a Newtonian fluid and considering the dope speed and needle diameter, this results in a shear rate of 130 s⁻¹ expressed by Equation (2).

$$γ = \frac{4 Q}{π {r}^{3}} \qquad ( 2 )$$

As discussed before, this shear rate was enough to achieve the terminal viscosity (465 mPa·s) by shear thinning, promoting a well oriented and aligned flow.

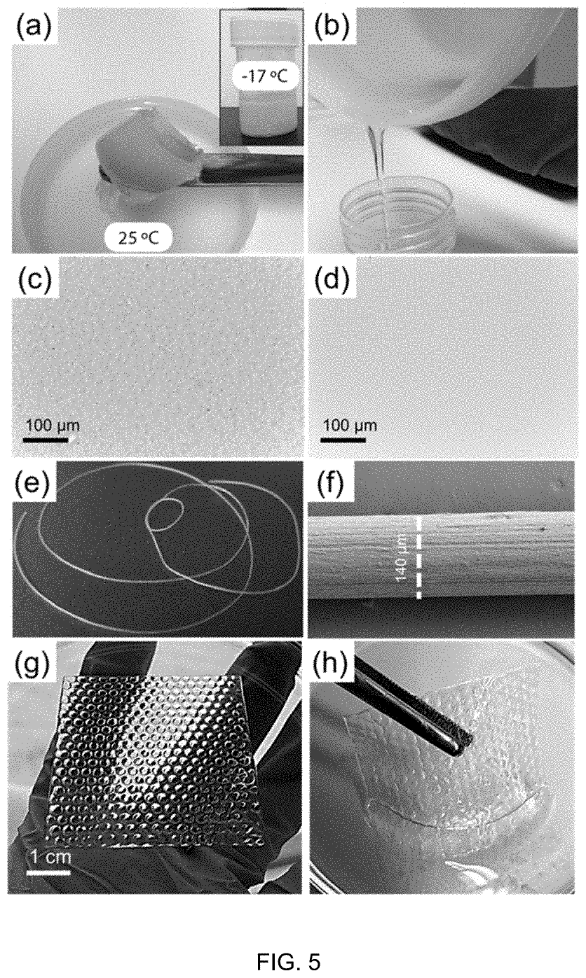

The dope was extruded into an acid bath (no air gap) with a composition of 10% H₂SO₄ w/w and 10% Na₂SO₄. This is a typical acid bath condition used in the Viscose process and used for similar dopes. After the acid bath, a second cross-linking bath containing 1% w/w ZnCl₂ at pH 2 was used to promote the cross-linking. The cross-linking bath was followed by three washing baths, the first containing Milli-Q water, the second containing ethanol, and the last containing Milli-Q water. The five baths were at room temperature and each contained 2 liters of the respective solution. The filament's residence time at each bath is 15 min. Finally, the well-washed filament is collected and cut into small pieces (about 1 m each) and dried under tension using magnets on a metal board. The final filament obtained is shown in FIG. 5(e).

FIGS. 5(a) to 5(f) illustrate the above described alkali soluble cellulose dope preparation. FIG. 5(a) shows the dope after thawing at room temperature, in the small square the appearance when frozen at −17° C. FIG. 5(b) shows the dope after thawing in a planetary centrifuge (2000 rpm, 20 min). FIG. 5(c) shows a light microscope image of the dope thawed without centrifugation. FIG. 5(d) shows the dope light microscope image after thawing under centrifugation. FIGS. 5(e) and 5(f) illustrate the 1D filament produced from alkali-soluble cellulose: FIG. 5(e) shows the cross-linked filament after drying, and FIG. 5(f) shows a lateral SEM image of the cross-linked filament at 250× magnification. FIG. 5(g) shows the 3D printed mesh from the alkali soluble dope in the size of 5 cm to 5 cm. FIG. 5(h) shows the self-standing 3D printed mesh after crosslinking in 1% w/w ZnCl₂ at pH 2 and washing step.

The spinned filaments comprise microcrystalline cellulose.

The filaments obtained exhibited a well-aligned structure as shown in the SEM images. Finally, the mechanical properties of these filaments were assessed and compared to the filaments without any cross-linking step. As shown in Table 3, the cross-linking procedure increases the resulting filament's toughness by one order of magnitude.

TABLE 3
Mechanical properties of spun regenerated cellulose
filaments in dry conditions. The values in parentheses
are standard deviations of the respective values.

| | | Tensile | Tensile | | |
|---|---|---|---|---|---|
| | Modulus | Strength | Strain | Toughness | Tenacity |
| Sample | [GPa] | [MPa] | [%] | [kJ · m⁻³] | [cN · Tex⁻¹] |
| Not cross- | 5.2 (2.0) | 41 (12) | 1.5 (0.6) | 230 (80) | 1.8 (0.6) |
| linked | | | | | |
| Cross- | 6.9 (0.9) | 70 (4) | 5.9 (1.9) | 3100 (1400) | 3.96 (0.2) |
| linked | | | | | |

The cellulosic gel, which is an intermediate in the preparation process described above, may be processed by spinning to produce one-dimensional filaments, or alternatively by 3D printing to produce three-dimensional meshes or complex structures.

The spinning process may be any of the following: wet spinning, dry jet wet spinning.

For example, the Viscose process may be used for preparing the cellulosic material in the form of a gel or fibres.

During wet spinning and in contact with the anti-solvent, the extruded filament shrinks to form a strong structure.

Wet-spun and dried fibres or filaments are particularly suitable for knitting. The dried cellulosic filaments may be knitted into a form of a mesh with auxetic design.

Alternatively, the cellulosic material in the gel form may be processed through a direct ink writing method or 3D printing to produce a 3D structure with auxetic design.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

The present invention is industrially applicable at least in support structures for mammalian tissues and organs and the manufacturing methods of such structures.

ACRONYMS LIST

| | |
|---|---|
| BNC | bacterial nanocellulose |
| CNF | cellulose nanofibril |
| TOCNF | TEMPO-oxidized cellulose nanofibril |
| POP | pelvic organ prolapse |
| SUI | stress urinary incontinence |
| PLA | polylactic acid |

### CITATION LIST

### Non Patent Literature

Fang-Fang Ai, Meng Mao, Ye Zhang, Jia Kang, Lan Zhu, Experimental study of a new original mesh developed for pelvic floor reconstructive surgery, Int. Urogynecol. J. (2020) 31:79-89.
Katrina M. Knight et al., Preventing Mesh Pore Collapse by Designing Mesh Pores With Auxetic Geometries: A Comprehensive Evaluation Via Computational Modeling, Journal of Biomechanical Engineering May 2018, Vol. 140, 051005-1.
Jonas, R.; Farah, L. F., Production and Application of Microbial Cellulose. Polym. Degrad. Stab. 1998, 59 (1-3), 101-106.
Wang, J.; Tavakoli, J.; Tang, Y., Bacterial Cellulose Production, Properties and Applications with Different Culture Methods— A Review. Carbohydr. Polym. 2019, 219 (May), 63-76.
Azeredo, H. M. C.; Barud, H.; Farinas, C. S.; Vasconcellos, V. M.; Claro, A. M., Bacterial Cellulose as a Raw Material for Food and Food Packaging Applications. Front. Sustain. Food Syst. 2019, 3 (February).
Ma, P.; Chang, Y.; Boakye, A.; Jiang, G., Review on the Knitted Structures with Auxetic Effect. J. Text. Inst. 2017, 108 (6), 947-961.
Knight, K. M.; Moalli, P. A.; Abramowitch, S. D., Preventing Mesh Pore Collapse by Designing Mesh Pores with Auxetic Geometries: A Comprehensive Evaluation Via Computational Modeling. J. Biomech. Eng. 2018, 140 (5), 1-8.
Dällenbach, P., To Mesh or Not to Mesh: A Review of Pelvic Organ Reconstructive Surgery. Int. J. Womens. Health 2015, 7, 331-343.
Bielecki, S.; Krystynowicz, E. A.; Marianna, Study on the Drug Loading and Release Potential of Bacterial Cellulose. Cellulose 2005, i (2), 37-46.
Badshah, M.; Ullah, H.; Khan, S. A.; Park, J. K.; Khan, T., Preparation, Characterization and in-Vitro Evaluation of Bacterial Cellulose Matrices for Oral Drug Delivery. Cellulose 2017, 24 (11), 5041-5052.
Badshah, M.; Ullah, H.; Khan, A. R.; Khan, S.; Park, J. K.; Khan, T., Surface Modification and Evaluation of Bacterial Cellulose for Drug Delivery. Int. J. Biol. Macromol. 2018, 113, 526-533. https://doi.org/10.1016/j.ijbiomac.2018.02.135.
Prawoto, Y., Seeing Auxetic Materials from the Mechanics Point of View: A Structural Review on the Negative Poisson's Ratio. Comput. Mater. Sci. 2012, 58 (June 2012), 140-153.
Ugbolue, S. C.; Kim, Y. K.; Warner, S. B.; Fan, Q.; Yang, C. L.; Kyzymchuk, O.; Feng, Y.; Lord, J., The Formation and Performance of Auxetic Textiles. Part II: Geometry and Structural Properties. J. Text. Inst. 2011, 102 (5), 424-433.
Ugbolue, S. C.; Kim, Y. K.; Warner, S. B.; Fan, Q.; Yang, C. L.; Kyzymchuk, O.; Feng, Y., The Formation and Performance of Auxetic Textiles. Part I: Theoretical and Technical Considerations. J. Text. Inst. 2010, 101 (7), 660-667.
Pan, K.; Fang, P.; Cao, B., Novel Composite Membranes Prepared by Interfacial Polymerization on Polypropylene Fiber Supports Pretreated by Ozone-Induced Polymerization. Desalination 2012, 294, 36-43.
Walzak, M. J.; Flynn, S.; Foerch, R.; Hill, J. M.; Karbashewski, E.; Lin, A.; Strobel, M., UV and Ozone Treatment of Polypropylene and Poly(Ethylene Terephthalate). J. Adhes. Sci. Technol. 1995, 9 (9), 1229-1248.
Salas, C.; Genzer, J.; Lucia, L. A.; Hubbe, M. A.; Rojas, O. J., Water-Wettable Polypropylene Fibers by Facile Surface Treatment Based on Soy Proteins. ACS Appl. Mater. Interfaces 2013, 5 (14), 6541-6548.
Wang, L.; Lundahl, M. J.; Greca, L. G.; Papageorgiou, A. C.; Borghei, M.; Rojas, O. J., Effects of Non-Solvents and Electrolytes on the Formation and Properties of Cellulose I Filaments. Sci. Rep. 2019, 9 (1), 1-11.
Lundahl, M. J.; Cunha, A. G.; Rojo, E.; Papageorgiou, A. C.; Rautkari, L.; Arboleda, J. C.; Rojas, O. J., Strength and Water Interactions of Cellulose I Filaments Wet-Spun from Cellulose Nanofibril Hydrogels. Sci. Rep. 2016, 6 (February), 1-13.
Kucharska, M.; Sikora, M.; Brzoza-Malczewska, K.; Owczarek, M., Antimicrobial Properties of Chitin and Chitosan. In Chitin and Chitosan; Broek, L. A. M. van den, Boeriu, C. G., Eds.; Wiley & Sons Ltd, 2019; pp 169-187.
Davidson, G. F. The dissolution of chemically modified cotton cellulose in alkaline solutions. Part I—In solutions of sodium hydroxide, particularly at temperatures below the normal. J. Text. Inst. Trans. 25, T174-T196 (1934).
Davidson, G. F. The dissolution of chemically modified cotton cellulose in alkaline solutions. Part III—In solutions of sodium and potassium hydroxide containing dissolved zinc, beryllium and aluminium oxides. J. Text. Inst. Trans. 28, T27-T44 (1937).
Davidson, G. F. The dissolution of chemically modified cotton cellulose in alkaline solutions. Part II—A comparison of the solvent action of solutions of lithium, sodium, potassium, and tetramethylammonium hydroxides. J. Text. Inst. Trans. 27, T112-T130 (1936).
Vehviläinen, M. Wet-spinning of cellulosic fibres from water-based solution prepared from enzyme-treated pulp. Tampere University of Technology. Publication vol. 1312 (2015).
Vehviläinen, M. et al. Dissolution of enzyme-treated cellulose using freezing-thawing method and the properties of fibres regenerated from the solution. Cellulose 22, 1653-1674 (2015).
Sobue, H., Kiessing, H. & Hess, K. The cellulose-sodium hydroxide-water system as a function of the temperature. Z Phys Chem B 43, 309-328 (1939).
Budtova, T. & Navard, P. Cellulose in NaOH-water based solvents: a review. Cellulose 23, 5-55 (2016).
Lundahl, M. J. et al. Strength and Water Interactions of Cellulose i Filaments Wet-Spun from Cellulose Nanofibril Hydrogels. Sci. Rep. 6, 1-13 (2016).
Lundahl, M. J., Klar, V., Wang, L., Ago, M. & Rojas, O. J. Spinning of cellulose nanofibrils into filaments: A review. Ind. Eng. Chem. Res. 56, 8-19 (2017).
Lundahl, M. J., Berta, M., Ago, M., Stading, M. & Rojas, O. J. Shear and extensional rheology of aqueous suspensions of cellulose nanofibrils for biopolymer-assisted filament spinning. Eur. Polym. J. 109, 367-378 (2018).
Väisänen, S. et al. Cellulose dissolution in aqueous NaOH—ZnO: cellulose reactivity and the role of ZnO. Cellulose 9, (2021).
Roy, C., Budtova, T. & Navard, P. Rheological properties and gelation of aqueous cellulose-NaOH solutions. Biomacromolecules 4, (2003).
Liebert, T., Schiller, F. & Jena, D. Cellulose Solvents—Remarkable History, Cellul. Solvents Anal. Shap. Chem. Modif. 3-54 (2010).

1. An auxetic structure comprising or consisting of cellulosic material.

2. The auxetic structure according to claim 1, comprising nanostructured or microstructured cellulose on at least one of its surfaces, or consisting of nanostructured or microstructured cellulose and optionally other cellulosic material.

3. The auxetic structure according to any of the preceding claims, comprising a mesh-like overall structure.

4. The auxetic structure according to any of the preceding claims, comprising a knitted or woven auxetic structure.

5. The auxetic structure according to any of the preceding claims, comprising a moulded auxetic structure having openings extending through the structure.

6. The auxetic structure according to any of the preceding claims, comprising a mesh or filaments made of a thermoplastic material and at least partially coated with microstructured or nanostructured cellulose, such as bacterial nanocellulose, and wherein preferably the thickness of the coating is at least 10 nm, such as in the range 20 to 500 nm, for example in the range 50 to 150 nm.

7. The auxetic structure according to any of the preceding claims, comprising cellulosic material, such as cellulose nanofibrils or cellulose filaments, obtained by a spinning process.

8. The auxetic structure according to any of the preceding claims, comprising filaments or a mesh obtained from a cellulosic material by a printing process, such as 3D printing or direct ink writing, preferably from a gel or a suspension comprising cellulosic material.

9. The auxetic structure according to any of the preceding claims, which structure has a negative Poisson's ratio and consequently exhibits lateral expansion when subjected to tension.

10. The auxetic structure according to any of the preceding claims, which structure comprises nanocellulose, such as wood-based nanocellulose or bacterial nanocellulose, and polyolefin, or consists of nanocellulose.

11. The auxetic structure according to any of the preceding claims, wherein the nanocellulose comprises or consists of cellulose produced by bacteria and in the form of nanofibrils.

12. The auxetic structure according to any of the preceding claims, wherein the nanocellulose comprises or consists of wood-based cellulose which has been disintegrated to nanostructured cellulose.

13. The auxetic structure according to any of the preceding claims, which structure comprises openings that are configured to expand under load.

14. The auxetic structure according to any of the preceding claims, which has a Young's Modulus of at least 700 MPa in wet condition.

15. The auxetic structure according to any of the preceding claims, which has tensile strength of at least 1.5 MPa in wet condition.

16. The auxetic structure according to any of the preceding claims for use in the treatment of pelvic organ prolapse, urinary incontinence, breast reconstruction, hernias, or fecal incontinence.

17. A support structure, comprising the auxetic structure according to any of the preceding claims, wherein said support structure is configured to support a tissue or an organ of a mammal, such as a human or a dog.

18. The support structure according to claim 17, which is a pelvic organ prolapse mesh, a urinary incontinence sling or tape for a man or a woman, a breast reconstruction support structure, a hernia mesh or a fecal incontinence support structure.

19. The support structure according to any of claims 17 to 18, which is a support mesh, a support sling or a support tape.

20. A method of preparing an auxetic structure, comprising:
forming an auxetic structure comprising or consisting of a cellulosic material, or
providing an auxetic guiding structure, and applying or growing a cellulosic material on at least a part of a surface of the guiding structure.

21. The method according to claim 20, wherein the cellulosic material comprises or consists of nanostructured and/or microstructured cellulose.

22. The method according to claim 20 or claim 21, wherein:
the auxetic guiding structure is prepared in a form of a guiding mesh by knitting from filaments or by printing.

23. The method according to claim 22, wherein said filaments comprise or consists of hydrophobic filaments, such as filaments made of a thermoplastic material, such as nylon, polypropylene, polyethylene, polyurethane, polycaprolactone, polyethylene terephthalate or a combination thereof.

24. The method according to claim 22, wherein said guiding mesh is prepared by means of a 3D printing method from a biodegradable thermoplastic material, such as polylactic acid.

25. The method according to any of claims 20 to 24, wherein
before said applying or growing step, said guiding structure is treated by a priming treatment, such as a hydrophilization treatment, an oxidizing treatment, a plasma treatment, or a protein adsorption treatment, and
as a result of said priming treatment, the bonding between the cellulosic material and a surface of the guiding structure is improved.

26. The method according to any of claims 20 to 22, wherein said auxetic guiding structure comprises hydrophilic filaments, such as filaments made of cellulose nanofibrils by a spinning process.

27. The method according to any of claims 20 to 26, wherein said applying or growing step comprises introducing a bacterial nanocellulose culture medium on a surface of said auxetic guiding structure, and allowing said culture medium to grow on said auxetic guiding structure.

28. The method according to claim 20, wherein said forming comprises:
dissolving a cellulosic material;
regenerating the dissolved cellulosic material to obtain a cellulosic gel or cellulosic fibres;
preparing the auxetic structure from said gel or fibres, preferably by knitting or printing.

29. The method according to claim 20, wherein said forming comprises moulding an auxetic structure of a cellulosic material comprising or consisting of nanostructured and/or microstructured cellulose.

30. The method according to claim 29, comprising:
providing a positive master mould exhibiting an auxetic shape,
preparing a negative mould from a thermoplastic polymeric material by using the positive master mould,
filling the negative mould with said cellulosic material comprising or consisting of nanostructured and/or microstructured cellulose, to prepare a corresponding positive structure, which is the auxetic structure.

31. The method according to claim 30, wherein said filling comprises applying a bacterial nanocellulose culture medium into said negative mould and allowing said culture medium to grow and fill the mould.

32. Use of a cellulosic material, such as nanostructured or microstructured cellulose, in a coating of or as a material of an auxetic structure configured for use as a support structure for a tissue or an organ of a mammal, such as a human.

33. A method of treating a mammal, comprising implanting a support structure according to any of claims 17 to 19 into a body of the mammal to support a tissue or an organ of the mammal.
